(19) United States
(12) Patent Application Publication
(10) Pub. No.: US 2021/0046473 A1
(43) Pub. Date: Feb. 18, 2021

(54) MULTIPLEXED MASS AND NANOPARTICLE DETECTION IMAGING, TOOLS, FLUIDICS, AND METHODS OF MAKING AND USING THE SAME

(71) Applicant: INBIOS INTERNATIONAL, INC., Seattle, WA (US)

(72) Inventors: James William NEEDHAM, Seattle, WA (US); Syamal RAYCHAUDHURI, Seattle, WA (US)

(21) Appl. No.: 16/994,171

(22) Filed: Aug. 14, 2020

Related U.S. Application Data
(60) Provisional application No. 62/887,297, filed on Aug. 15, 2019.

(51) Int. Cl. B01L 3/00; G01N 33/58; G01N 21/45

(52) U.S. Cl. CPC B01L 3/5027; G01N 33/587; B01L 2300/0636; B01L 2200/04; G01N 21/45

(57) ABSTRACT

This disclosure generally describes to products and compositions relating to a modified Interferometric Reflectance Imaging Sensor (IRIS) platform, wherein said modification relates to, among other things, one or more of a fluidics pathway, a cassette housing for a chip cartridge, a diluted biological specimen and properly labeled gold nanoparticles observed in a wide field-of-view (i.e., not single nanoparticle counting). The present disclosure also includes products and methods that may be used separately or together with the modified IRIS platform, including one or more of a chip spotted with a unique population of purpose-specific test materials, the test materials themselves, a chip cartridge casing, and compositions for use in a fluidics pathway. As contemplated herein, the products and methods disclosed herein may be used for myriad applications, including assessment of overall health, immune profiling abnormal health conditions, including infections and diseases, such as cancer, etc. Data generated by the products and processes described herein may also be suitable for use with automated analysis including typical machine learning tools.

### FIELD OF THE INVENTION

This disclosure generally describes to products and compositions relating to a modified Interferometric Reflectance Imaging Sensor (IRIS) platform, wherein said modification relates to, among other things, one or more of a fluidics pathway, a cassette housing for a chip cartridge, a diluted biological specimen and properly labeled gold nanoparticles observed in a wide field-of-view (i.e., not single nanoparticle counting). The present disclosure also includes products and methods that may be used separately or together with the modified IRIS platform, including one or more of a chip spotted with a unique population of purpose-specific test materials, the test materials themselves, a chip cartridge casing (enclosure), and compositions for use in a fluidics pathway. As contemplated herein, the products and methods disclosed herein may be used for myriad applications, including assessment of overall health, abnormal health conditions, including immune profiling, infections and diseases, such as cancer, etc. Data generated by the products and processes described herein may also be suitable for use with automated analysis including typical machine learning tools.

### BACKGROUND OF THE INVENTION

Various iterations of the Interferometric Reflectance Imaging Sensor (IRIS) platform and methods of use are known.

### BRIEF SUMMARY OF THE INVENTION

The present disclosure generally relates to products and methods for profiling biological specimens (serum, plasma, whole blood, urine, sputum, etc.) using a modified IRIS platform, wherein said modification relates to one or more of a fluidics pathway, a chip cartridge casing, a diluted biological specimen and properly labeled gold nanoparticles observed in a wide field-of-view (i.e., not single nanoparticle counting). Also included in the present disclosure are products and methods that may be used separately or together with the modified IRIS platform, including one or more of a chip spotted with a unique population of purpose-specific test materials, the test materials themselves, a chip cartridge casing (enclosure), and compositions for use in a fluidics pathway. The products and methods disclosed herein may be used for myriad applications, including to survey or detect indicia of overall health, characterize abnormal or unhealthy conditions, immune profiling, identify specific infections, diseases, cancer(s), etc. The products and methods described herein may further include the use of computer software and/or the use of machine learning to analyze the complex set of data results.

There are a number of key advantages when using the modified IRIS platform and processes as described herein. First, a multitude of binding data and characteristics may be observed for any one given specimen. For example, multiplexing dozens of potential targets for Lyme with the potential to add targets for other, unrelated diseases (e.g., HIV, hepatitis, etc.) becomes trivial as relevant reagents only need to be additionally spotted on the existing chip surface. Second, because reactions may be observed in real-time, temporal data may also be of significant value as second-by-second binding information is collected—not just end point values. Such temporal data may provide, for example, evidence of sample avidity and concentration. Third, the assay time is short as a test specimen may be actively injected over the chip surface; thus, ameliorating localized analyte depletion and reducing incubation times. Fourth, because the raw data is, itself, a series of sequentially collected images (an “image stack”), machine learning may be directly applied to either or both of the mass accumulation events and any of the subsequent gold nanoparticle binding events. This may further aid in the development of a sophisticated analytical tools to ultimately determine and interpret the specimen results: for instance, specific binding events for mass accumulation followed by specific IgM binding and specific IgG accumulation may be independently monitored to provide an overall status of the patient specimen. Fifth, the products and processes disclosed herein may incorporate gold nanoparticle detection without disrupting the stability of the reagents.

In some embodiments, the disclosed compositions and methods provide the ability to screen a single serum specimen against a multiplicity of targets spotted on a chip and to separately detect mass binding and, optionally, amplified specific signal with gold nanoparticle conjugates.

In some embodiments, the disclosed compositions and methods may take advantage of the dual modality of direct mass detection and enhanced signal derived from traditional lateral flow immunoassay gold nanoparticle technology.

Certain aspects of embodiments disclosed herein by way of example are summarized below. It should be understood that these aspects are presented merely to provide the reader with a brief summary of certain forms of an invention disclosed and/or claimed herein might take and that these aspects are not intended to limit the scope of any invention disclosed and/or claimed herein. Indeed, any invention disclosed and/or claimed herein may encompass a variety of aspect that may not be set forth below.

### DESCRIPTION OF FIGURES

The foregoing summary, as well as other features, aspects, and advantages of the present invention and the following detailed description of embodiments of the invention, will be better understood when read in conjunction with the claims and the appended drawings of an exemplary embodiments, wherein:

FIG. 1A illustrates a chip.

FIG. 1B illustrates a flow cell assembly.

FIG. 1C illustrates the loaded chip in an early generation IRIS prototype instrument.

FIG. 2A illustrates how light reflects from the top and bottom of the silicon/silicon-oxide chip surface.

FIG. 2B illustrates that as material binds to the surface, the intensity of the electromagnetic wave shifts.

FIG. 2C illustrates an example ‘difference image’ for the signal proportional to the mass of bound analyte with a variety of spotted ligands (unique ligands were spotted in triplicate).

FIG. 3 illustrates the direct detection (mass accumulation) of lethal toxin and lethal factor antigens with additional antibody “sandwiching” steps to determine antibody complementarity. Representative images (“slices”) from the collected data are also illustrated.

FIG. 4 illustrates serial 4-fold titrations for two different analytes (dengue NS1 and Y. pestis F1) proteins. Limits of detection of purified analyte in buffer approached 2 ng/mL for each analyte (3 standard deviations above the negative control spotted ligands).

FIG. 5 illustrates the direct binding of a 1:100 diluted Zika positive serum specimen in n=5 replicate chips. The anti-human IgM capture antibody consistently binds the diluted human IgM present in the serum sample while significant mass accumulation occurs at the spotted Zika NS1 antigen locations—potentially indicative of human antibody binding to the target antigen.

FIG. 6 illustrates the detection of 1 ng/mL of lethal factor (LF) antigen is significantly amplified upon the injection of a gold conjugated particle that also targets (LF). Red arrows indicate the injection of LF (1 ng/mL), gold nanoparticles and buffer only.

FIG. 7A illustrates the direct mass binding of a normal human serum specimen.

FIG. 7B illustrates the same chip from FIG. 7A is then injected with gold nanoparticles targeting human IgM and human IgG. The total assay time is about 35 minutes. Red arrows indicate the injections of 1:100 diluted serum, gold nanoparticles that target human IgM, and gold nanoparticles that target human IgG. The vertical red “bands” indicate the end point of the incubation time and may be used for estimating end-point values.

FIG. 8A illustrates the direct mass binding of a Lyme positive human serum specimen.

FIG. 8B illustrates the same chip from FIG. 8A is then injected with gold nanoparticles targeting human IgM and human IgG. The total assay time is about 35 minutes. Red arrows indicate the injections of 1:100 diluted serum, gold nanoparticles that target human IgM and gold nanoparticles that target human IgG. The vertical red “bands” indicate the end point of the incubation time and may be used for estimating end-point values.

FIG. 9 illustrates the mass binding of the St. Luke's #2 specimen to the VoVo antigen is markedly stronger—with signals exceeding about 600 AU in under 10 minutes.

FIG. 10 illustrates two different cartridges that may hold the IRIS chip.

### DETAILED DESCRIPTION OF THE INVENTION

The present disclosure generally relates to products and methods for profiling serum, e.g., blood serum, using a modified IRIS platform, wherein said modification relates to one or more of a fluidics pathway, a chip cartridge, a cassette housing for the chip cartridge and properly labeled gold nanoparticles observed in a wide field-of-view (i.e., not single nanoparticle counting). Also included in the present disclosure are products and methods that may be used separately or together with the modified IRIS platform, including one or more of a chip spotted with a unique population of purpose-specific test materials, the test materials themselves, stabilizing a spotted chip surface, a chip cartridge casing, and compositions for use in a fluidics pathway. The products and methods disclosed herein may be used for myriad applications, including to observe and make use of indicia of overall health, immune profiling, characterize abnormal or unhealthy conditions, identify specific infections, diseases, cancer(s), etc. The products and methods described herein may further include the use of computer software and/or the use of machine learning to analyze data results.

In one embodiment, the present disclosure relates to an apparatus comprising: an interferometric based imaging sensor (e.g., IRIS); multiple distinct target compatible materials bound to a functionalized chip surface (e.g., a microarray of spots using target analytes such as peptides, proteins and nucleic acids); a stabilized chip surface and fluidic cartridge, wherein said cartridge (after proper treatment) may be stored at room temperature for an extended period of time; the stabilized fluidic cartridge is subsequently placed in contact with the fluidics system; a cassette housing (e.g., injection molded plastic holder) to manipulate and label an individual fluidic cartridge while protecting the optically sensitive fluidic cartridge; and the use of stabilized and/or lyophilized conjugate nanoparticles for subsequent signal detection in the imaging sensor platform. It is noted that the present disclosure includes methods for room temperature stabilizations. For example, the cartridge may be stabilized for desiccated room temperature storage for several months or years. Further, it is noted that a labeled gold formulation for protein A that is room temp stable for about 18 months is possible. Further, gold nanoparticles may be lyophilized for use with the apparatus, systems, and methods described herein.

Apparatuses described herein may be used by, for example, following one or more of the following steps: preparing a heterogeneous biological sample (e.g., serum, plasma, whole blood, sputum, urine, pus) by diluting the biological sample in an appropriate buffer; contacting the sample with the chip; and imaging material mass accumulation resulting from binding between at least one of the multiple distinct target compatible materials and at least one component of the sample.

One method of using an apparatus as described herein, comprises one or more of the following steps: subsequently injecting labeled nanoparticles after the observed mass accumulation (e.g., spherical gold nanoparticles or latex spheres) in a wide field-of-view to observe reflection or scattering of light from the average accumulation of nanoparticles at localized spots on the chip surface (i.e., not single nanoparticle counting); sequentially injecting uniquely labeled nanoparticles (e.g., spherical gold nanoparticles or latex spheres that provides separate, obtaining step-wise information about the specimen status using a wide field-of-view modality; and simultaneously injecting diluted biological samples (e.g., serum, plasma, whole blood, sputum, urine, pus) pre-mixed with labeled nanoparticles using a wide field-of-view modality. It is noted that wide field-of-view observation is distinct from single nanoparticle counting. Further, obtaining step-wise information about the specimen status using a wide field-of-view modality is important as, for example, information first relating to IgM and then IgG gives significant diagnostic information. Also possible is the simultaneous injection of diluted biological samples (e.g., serum, plasma, whole blood, sputum, urine, pus) pre-mixed with labeled nanoparticles using a wide field-of-view modality. Such a simultaneous injection may be a method for cutting the assay time, for example, to about 20 to about 25 minutes total.

In certain embodiments, the present disclosure relates to a room temperature stabilized fluidic cartridge system.

In certain embodiments, the present disclosure relates to appropriate buffers for diluting and running specimens.

In certain embodiments, the present disclosure relates to step-wise injections of specimen and labeled nanoparticles in order to detect sequentially useful information about the specimen (e.g., IgM antibody content followed by IgG antibody content).

In certain embodiments, the present disclosure relates to the use of disease specific analytes (nucleic acids, peptides, proteins and antibodies) to detect direct mass accumulation followed by more specific step-wise conjugate nanoparticles to indicate disease status (e.g., anti-Human IgM gold nanoparticles and anti-Human IgG gold nanoparticles) for real-time and endpoint binding measurements.

In certain embodiments, the present disclosure relates to the step-wise detection of the specimen binding events: mass binding followed by conjugated nanoparticle binding in a wide field-of-view detection modality.

In certain embodiments, the present disclosure relates to a room temperature stabilized and/or lyophilized conjugate nanoparticle system to be incorporated with the step-wise detection of the specimen binding events for the detection of antibody isotype.

In certain embodiments, the present disclosure relates to a room temperature stabilized and/or lyophilized conjugate nanoparticle system to be incorporated with the step-wise detection of the specimen binding events for the detection of specific antigen(s) or nucleic acids that are present in the specimen.

In certain embodiments, the present disclosure relates to a series of positive and negative control analytes to indicate the presence of the diluted specimen (e.g., anti-Hu IgM and anti-Hu IgG) and to monitor for non-specific binding (e.g., an unrelated nucleic acid sequence, protein or antibody).

In certain embodiments, the present disclosure relates to methods for using any of the embodiments disclosed herein for imaging material mass accumulation followed by more specific gold nanoparticle accumulation observed in the wide-field (i.e., not nanoparticle counting).

In one embodiment, the present disclosure relates to an apparatus comprising one or more of the following features: multiple distinct target compatible materials bound to a chip, wherein said materials target tick borne infections (e.g., Lyme, Babesia, etc.); multiple distinct target compatible materials bound to a chip, wherein said materials target flavivirus and alphavirus infections (dengue, zika, chikungunya, etc); multiple distinct target compatible materials bound to a chip, wherein said materials target flu, HIV-1, HIV-2, HBV, and HCV; multiple distinct target compatible materials bound to a chip, wherein said materials target known allergens; a series of target analytes specific for Lyme infection including VoVo, VoBop, Vo4, OspC variants, DbpA, DbpB, VIsE, FlaB and C6 bound to the chip surface; a series of target analytes specific for Babesia infection including BMSA1 and BMN1-17 bound to the chip surface; a series of target analytes specific for dengue and Zika infection including dengue NS1 and envelope proteins; a series of antibodies targeting dengue NS1 for the detection of circulating dengue NS1 in a patient specimen including 528.1133, 528.292, 528.1299, 1010.511 and 1010.522; and a series of target analytes specific for chikungunya infection, including chikungunya envelope proteins.

In certain embodiments, the present disclosure relates to an analysis method comprising one or more of the following: analysis of binding events that may incorporate dynamic or endpoint measurements; cut-off thresholds to determine positivity based upon one or multiple target binding events to a target; cut-off thresholds that incorporate one or multiple targets; cut-off thresholds that incorporate measurements from mass accumulation and step-wise addition of conjugated nanoparticles, either individually or jointly; and machine learning applied to categorizing specimens (e.g., positive or negative, IgM positive, antigen positive, acute, convalescent, etc.) based upon the real-time or endpoint measurements with mass and/or step-wise addition of conjugated nanoparticles where a training set is used to train and properly classify specimens according to the desired status.

IRIS

The lnterferometric Reflectance Imaging Sensor (IRIS) was developed as a unique analytical tool for the simultaneous direct detection of analyte-ligand binding pairs (e.g., DNA-DNA hybridization, antibody-antigen binding, etc.). The IRIS technique relies upon the simple and robust principle of the interference of electromagnetic waves at a thin surface. Normally incident light (from a multicolored LED source) illuminates the chip through a flow cell. The reflected light is captured by a scientific camera. Sequential images are recorded, and as mass accumulates at any given spot, the reflected light wave is ‘shifted.’ These shifts are proportional to the amount of mass that accumulates at a given spot. Images are collected ‘real time’ and the binding as a function of time is directly monitored. As the spots (i.e., microarray) may represent a multitude of targets, this permits the simultaneous monitoring of binding to multiple targets allowing a specimen profile to be determined.

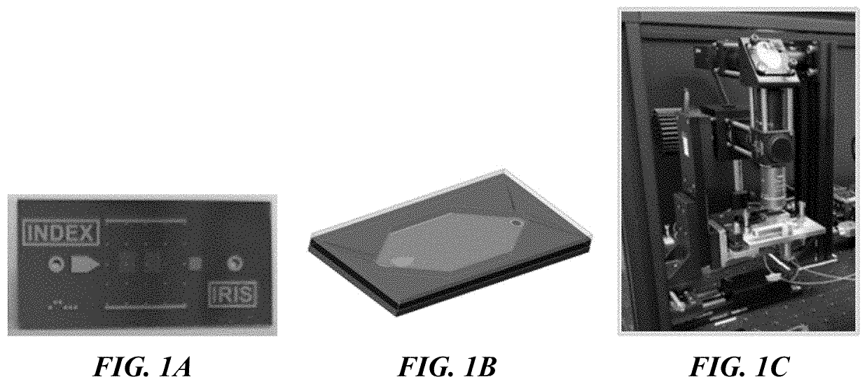

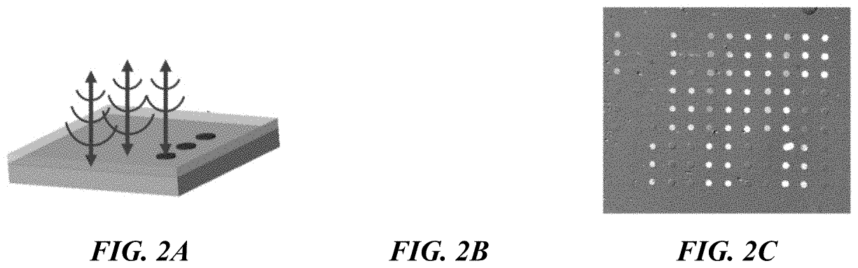

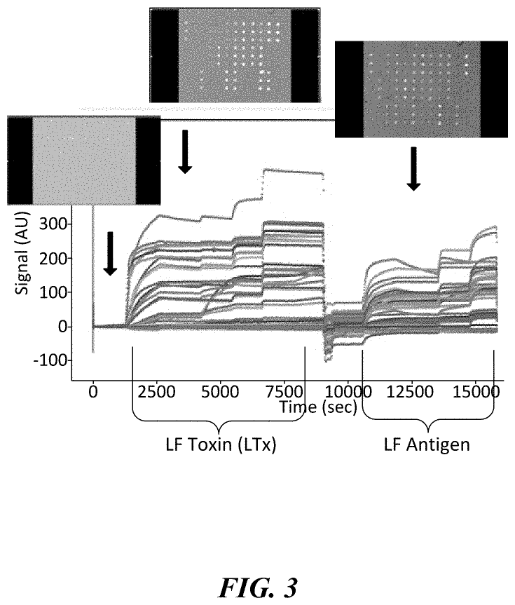

Exemplary IRIS components and explanatory images are provided herein as FIG. 1A, FIG. 1B, FIG. 1C, FIG. 2A, FIG. 2B, and FIG. 3C. used in IRIS. FIG. 1A illustrates a chip. FIG. 1B illustrates a flow cell assembly. FIG. 1C illustrate the loaded chip is in an early generation IRIS prototype instrument. FIG. 2A illustrates light that reflects from the top and bottom of the silicon/silicon-oxide chip surface. FIG. 2B illustrates that as material binds to the surface, the intensity of the electromagnetic wave shifts. FIG. 2C illustrates an example ‘difference image’ for the signal proportional to the mass of bound analyte with a variety of spotted ligands (unique ligands were spotted in triplicate).

The present disclosure generally relates to specific applications and uses of IRIS comprising particular fluidics, fluidic cartridges, and/or related opto-mechanics for analysis of test materials. As described herein, the IRIS system directly visualizes the chip surface through the flow cell, light that is reflected from any particle may also be directly observed.

The present disclosure also relates to potential applications of IRIS that take advantage of the dual modality of direct mass detection in tandem with traditional lateral flow immunoassay gold nanoparticle technology. That is, in some embodiments, the present disclosure relates to the direct detection of mass binding to the sensor surface and the subsequent detection of labeled gold nanoparticles. For example, target antigens, antibodies and nucleic acids are spotted down onto a functionalized IRIS chip using existing microarray technologies. Fluidic handling is introduced into the machine to properly prime and load diluted patient specimens, such as serum. A cartridge holds and protects the IRIS chip until use. The patient specimen binds to the various targets on the chip, localized thickness increases (mass accumulations) are visualized in the IRIS system. A patient serum assessment or profile is then be generated. In certain embodiments, the profile is not considered diagnostic or prognostic, but rather as providing a pattern of reactivity that can be subsequently interpreted (either by a certain analysis threshold or machine learning) to indicate if a patient should undergo follow up testing. In some embodiments, simultaneous screening for binding reactivity to Lyme disease (or other tick borne infections), Chagas disease, flavivirus antigens, HIV and hepatitis may indicate to the medical practitioner that follow up testing is necessary (particularly with infections that may have limited symptoms during the initial infection but may present with severe chronic symptoms in chronic stages of the disease). In exemplary embodiments, the present disclosure provides advantages for the detection of infections, such as Lyme infection.

Fluidics

Patient samples are collected from hospitals, labs, biobanks, blood centers, and other clinical organizations. In embodiments, the samples comprise biofluids, solid tissues, cells, or a combination thereof. In some embodiments, the biofluids comprise whole blood, plasma, serum, buffy coats, urine, stool, CSF, liquid cytology, swabs, saliva, sputum, or a combination thereof. The further embodiments, the biofluids are fresh or frozen and with our without additives, such as EDTA, Lithium, Heparin, and Sodium Citrate. In embodiments, the solid tissues comprise fresh, frozen, or fixed tissues. In embodiments, the cells are fresh or cryopreserved. In some embodiments, the cells are isolated from bone marrow, peripheral blood, and cord blood. In further embodiments, the cells comprise mononuclear cells, myeloid cells, lymphoid cells, and pluripotent cells. In specific embodiments, the patient sample is serum.

In embodiments, the patient samples are processed to collect proteins, peptides, nucleic acids, antibodies, lipids, polysaccharides, or a combination thereof. The samples may be prepared and processed with a standard protocol. In some embodiments, the samples size ranges from about 5 μl to 1,000 μl. In specific examples, the samples size ranges from about 10 μl to 20 μl. In some embodiments, the samples are diluted from about 1:1, 1:2, 1:5, 1:10, 1:20, 1:50, 1:100, 1:200, 1:500, or 1:1000 dilutions.

In specific embodiments, the serum is diluted at about 1:100 dilution. In some embodiments, the samples are flushed through filters ranging from about 10 μm to 40 μm. In specific embodiments, the samples are flushed through filters of about 22 μm.

In some embodiments, the samples are loaded onto the IRIS chip by a fluidic handling method that properly primes and loads the sample. In embodiments, the fluidic handling method comprises a fluidics pathway. In some embodiments, the fluidics pathway comprises a valve selector, de-bubbler, peristaltic pump, or a combination thereof. The valve selector allows for changing selection of fluids without disturbing the experimental process. In embodiments, the fluidics pathway further comprises a one way or a recycle-flow of the diluted patient sample. The one way fluidics pathway has a waste collector that collects all the flow through from the IRIS chip. The recycle flow fluidic pathway collects the sample after flow through and reloads the sample on the IRIS chip one or more times.

Chip, Cartridge Assembly, and Spotting

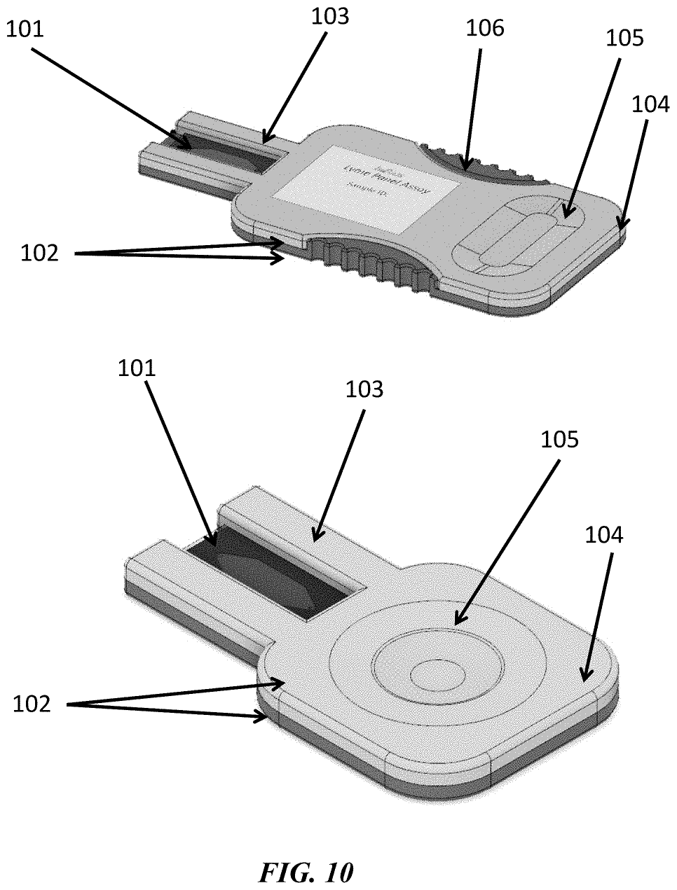

In some embodiments, the size of the chip (FIG. 1B and FIG. 10, reference 101) is the standardized size of 25 mm in length and 12.5 mm in length. Substrates of interest are spotted, for example, in triplicates on the chip according to standard protocol with a bench top spotter. In some embodiments, the substrates of interest comprise peptides, proteins, nucleic acids, carbohydrates, lipids, or fragments thereof. After spotting the substrates onto the chip, it is incubated at room temperature (15-30° C.) overnight.

Subsequently the chip is blocked and stabilized in formulated blocking solution. This solution contains 20% sucrose (w/v) and 5% goat serum in a phosphate buffered saline solution. Varying sucrose concentrations provide varying levels of stabilization and this is a necessary component for providing room temperature stability to the chip surface. The chip is blocked for 30-90 minutes. The blocked chip is dried, sealed, and stored at room temperature under desiccating conditions. Subsequently, the spotted chips may be sealed in a protective foil wrapper, which allows the spotted chip to be stably stored at room temperature for an extended period of time.

Due to the small size of the chip, it is often held with tweezers during the spotting, sample loading, and imaging processes. Holding the chip with tweezers is very unstable and difficult. As a result, the chip often slips from the tweezers and breaks. The present disclosure provides a casing to, among other things, ease handling and to protect and secure the chip during all experimental processes.

In some embodiments, the chip is held in a cartridge during the spotting, sample loading, and imaging processes. In some embodiments, the chip is spotted prior to insertion or inclusion in a cartridge.

In some embodiments, the cartridge is plastic. In some embodiments, the cartridge is disposable or reusable. In some embodiments, the reusable plastic casing is autoclavable or may be sterilized by other means.

The cartridge comprises two mated sides (102) which are substantially identical. Each mated side comprises a set of two parallel arms (103) and a body (104), as shown in FIG. 10. The parallel arms on both sides of the cartridge securely hold the chip in place. In embodiments, the arms are from about 8.5 mm to 12 mm apart, forming a width that allows the chip to be visualized by the IRIS. In preferred embodiments, the arms are about 11 mm to 11.5 mm apart. In embodiments, the arms are from about 20 mm to 30 mm in length. In preferred embodiments, the arms are from about 24 mm to 26 mm in length. In embodiments, the arms are from about 5 mm-20 mm in width. In preferred embodiments, the arms are about 8 mm in width.

The arms of the cartridge extend from the body. In embodiments, the body is from about 25 mm to 65 mm in length. In embodiments, the body is from about 35 mm to 40 mm in length. In other embodiments, the body is from about 55 mm to 60 mm in length. In further embodiments, the body comprises non-slip ridges (106) to facility gripping the cartridge by, for example, gloved or ungloved hands.

Mass Detection

The direct detection of mass accumulating at a defined geometry is part of the class of so-called ‘label free’ detection methods which includes surface plasmon resonance (SPR), biolayer interferometry (BLI) and others. Recent improvements to the IRIS design and procedures have improved the limits of detection of analyte in solution to single ng/mL levels—matching or exceeding published limits acquired via other methods. The real time binding can be monitored to establish kinetic rate information for purified analytes (i.e., antibody, antigen, DNA, etc.) or to detect binding of serum proteins (i.e., diluted human serum specimens) to target analytes. Binding signals are recorded at each individual spot by monitoring the intensity within the spot minus a nearby reference region as the control.

Any materials useful for spotting using conventional IRIS technology may be used with the products and processes described herein. Additionally, certain proprietary analytes such as the following may be included separately or in combination with each other or any other non-proprietary materials. Non-exclusive exemplary proprietary analytes that may be included as part of the products and processes described herein include, but are not limited to: WB123 (Filaria); 1010.511 (dengue); 1010.522 (dengue); 528.1133 (dengue); 528.292 (dengue); 5121.532 (dengue); BMSA1 (Babesia); BMN1-17; 62C1 (malaria); K39 (V. leishmania); and C11C (Cutaneous leishmania). Additional analytes that may be included as part of the products and processes described herein include, but are not limited to: DbpA (Lyme); DbpB (Lyme); BmpA (Lyme); VIsE (Lyme); FlaB (Lyme); VoVo (Lyme); VoBop (Lyme); Vo4 (Lyme); OCA (Lyme); OCB (Lyme); OCK (Lyme); and OCN (Lyme).

Examples of the direct detection of purified analyte with subsequent antibody sandwiching steps are shown in FIG. 3. Lethal toxin is first injected, followed by buffer and subsequent sandwiching antibodies. The chip was then eluted and lethal factor antigen was injected, followed by buffer and the additional subsequent antibody steps. Representative images (“slices”) from the collected data are also illustrated.

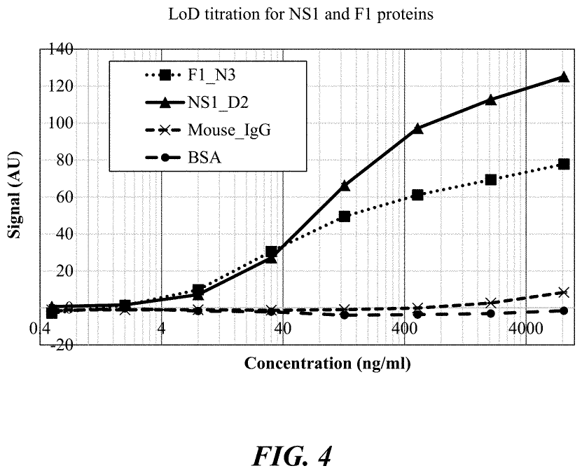

An exemplary limit of detection assay for purified analyte is shown in FIG. 4. Here, purified dengue NS1 and Y. pestis F1 were injected in serially increasing 4-fold concentrations (Needham J., 2019). Limits of detection of purified analyte in buffer approached 2 ng/mL for each analyte, which is 3 standard deviations above the negative control spotted ligands.

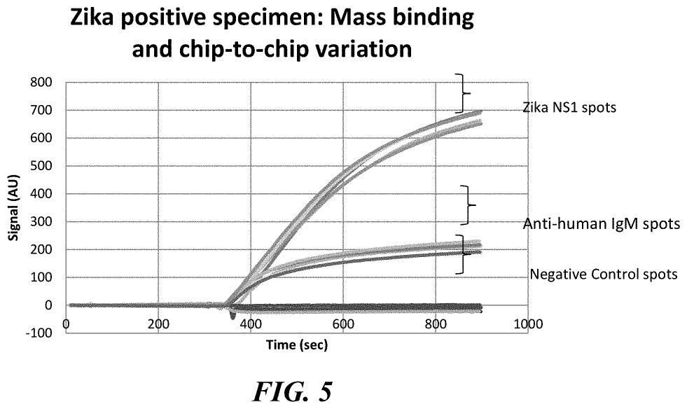

It is desirable to evaluate how diluted human serum binds to antigens that have been directly spotted onto the chip surface. To determine whether potential antibody binding (isotype independent) is significant and directly observable, five (5) separate chips were spotted with a series of Zika NS1 and control analytes (including an anti-human IgM antibody). The same diluted Zika positive serum specimen (1:100) was injected into each of these five chips in order to (1) monitor for direct binding of analyte to the spotted Zika NS1 (2) evaluate the capture of human IgM from the diluted serum specimen and (3) monitor the preliminary chip-to-chip variability. The results recorded from these chips are shown in FIG. 5 where specific binding to the Zika NS1 antigen is observed, IgM binding to the anti-human IgM capture antibodies is seen and negative control spots remain quiescent.

Sample Amplification

The IRIS modality is unique compared to other platforms due to the intrinsic imaging nature of the platform (i.e., microarray imaging is inherently a part of the instrument design) and the fact that the normally incident light passes through the chamber, giving an exquisitely sensitive detection to the binding of nanoparticles. In embodiments, the nanoparticles are inert. In some embodiments, the inert nanoparticles comprise gold, silver, platinum, or a combination thereof. In specific embodiments, the inert nanoparticles are gold. In some embodiments, the nanoparticles comprise a diameter ranges from about 10 nm, 20 nm, 30 nm, 40 nm, 50 nm, 60 nm, or a combination thereof. In specific embodiments, the nanoparticles comprise a diameter of about 40 nm.

The unique scattering properties of gold nanoparticles permit the digital detection of single virus and gold nanoparticles using the appropriate optical setup (Avci, Ünlü, Özkumur, & Ünlü, 2015). Standard lateral flow immunoassay (LFI) gold conjugate techniques are used to specifically conjugate and functionalize nanoparticles, e.g., such as anti-human IgM and anti-human IgG.

That is, in some embodiments, gold nanoparticles are directly labeled with a ligand, e.g., such as an antibody targeting IgG or IgM, and these gold nanoparticles are then specifically associated with bound analyte. In this manner, the mass binding events that are observed during an IRIS test may be confirmed as specific antibody subtypes by the addition of gold nanoparticles targeting those classes.

Additionally, due to the real-time monitoring of the binding events, it is possible to sequentially inject each gold conjugate to determine specimen binding due to any given analyte individually. That is, after the detection of direct mass accumulation at any given spot, gold nanoparticles can be injected that first target human IgM and specifically record binding to human IgM molecules followed by an injection of gold nanoparticles that target human IgG and specifically record binding to human IgG molecules.

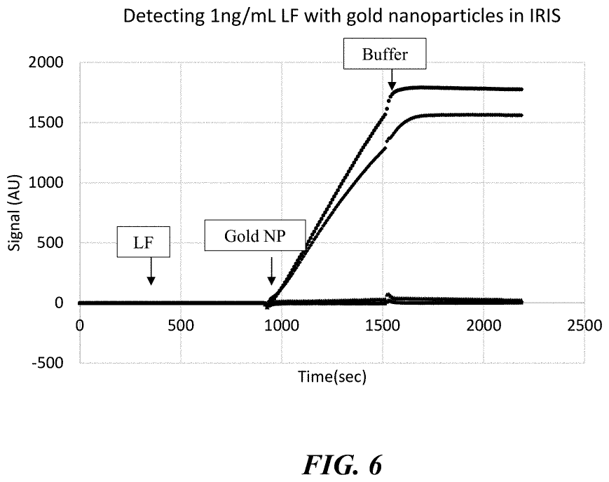

FIG. 6 illustrates the amplification of 1 ng/mL of lethal factor (LF) antigen by incorporating gold nanoparticles that also targets LF into the flow cell. The arrows indicate the injection of LF (1 ng/mL), gold nanoparticles and buffer, respectively.

In one embodiment, an LF antigen is injected for 10 minutes followed by the gold nanoparticles for an additional 10 minutes. As no secondary antibody is required for the detection of mass accumulation, the direct measurements are determined immediately as the specimen enters the chamber.

In some embodiments, the gold nanoparticles are conjugated to molecules. The gold conjugated molecules comprise peptides, proteins, nucleic acids, carbohydrates, lipids, or fragments thereof. In further embodiments, the gold conjugated molecules may comprise human IgG, human IgM, or a combination thereof.

It is noted that current multiplexed, array-based techniques may be utilized for detecting binding events (i.e., microarrays, microarray analyzers), but that many of these techniques require the use of fluorescently labeled antibodies and antigens and often suffer from photobleaching or ‘quenching’ of the signal.

Preliminary stability testing of the IRIS chip itself indicates room temperature stability (in a desiccated environment) is possible. Additionally, gold nanoparticle conjugates have been developed with lateral flow immunoassay techniques to create dried, room temperature stable conjugates (stable for years). Due to the intrinsic light scattering nature of gold nanoparticles, there is also no photobleaching or ‘quenching’ of the signal that is often a concern of fluorescently labeled antibodies and antigens.

### Application

The invention of the present disclosure provides many key advantages. The present IRIS platform as a diagnostic tool a multitude of binding data and characteristics are observed for any one given specimen. It can multiplex dozens of potential targets for specific diseases and targets for unrelated diseases. In embodiments, the diseases comprise, but are not limited to, various types of cancer, infectious diseases, genetic conditions, immunological diseases, heart diseases, allergies, or a combination thereof. In further embodiments, the infectious diseases comprise Lyme disease, tick borne infections, Zika virus, Chagas disease, flavivirus, HIV, hepatitis, or a combination thereof. In some embodiments, the specific disease is Lyme disease and the potential added targets for unrelated diseases are HIV, hepatitis, or a combination thereof. For multiplex detection of targets with the present IRIS platform, a multitude of disease substrates are spotted on the existing chip surface.

In some embodiments, the products and methods disclosed herein provide for short assay times, e.g., about 30 minutes, to test for mass binding, specific IgM and specific IgG signals. For example, in some embodiments, data for normal human serum and Lyme positive serum specimens may correlate to expected values obtained via ELISA. In other exemplary embodiments, data for other infectious disease types (e.g., Zika) may similarly correlate to results obtained with ELISA testing.

In a specific embodiment, this disclosure relates to analyte-ligand binding pairs (e.g., DNA-DNA hybridization, antibody-antigen binding, etc.). In the following sections we describe in some detail the principle of operation, the direct detection of mass binding to the sensor surface and the subsequent detection of labeled gold nanoparticles.

The temporal data collected by IRIS and processed by machine learning, along with the end point values determined, may provide binding information as a function of time and further provide real-time information regarding the sample avidity.

The assay time for the IRIS immunoassay is fast—due to the fact that the specimen is actively injected over the chip surface, localized analyte depletion may be ameliorated and incubation times reduced. For instance, in FIG. 6, the LF antigen is injected for 10 minutes followed by the gold nanoparticles that targets LF for an additional 10 minutes. As no secondary antibody is required for the detection of mass accumulation, the direct measurements are determined immediately as the specimen enters the chamber (as seen in FIG. 5).

The raw data is a series of sequentially collected images (an “image stack”), machine learning may be directly and naturally applied to both the mass accumulation events and gold nanoparticle binding events. This may further aid in the development of a sophisticated diagnostic algorithm to ultimately determine and interpret the specimen results independent of the end user.

In some embodiments, the binding of molecules of interest in the samples to the spotted substrates on the chip are visualized by the IRIS and stored as data. The molecules in the samples comprise peptides, proteins, nucleic acids, carbohydrates, lipids, fragments of any of the above, or a combination of any of the above. As the molecules bind to the substrates, machine learning processes the IRIS data. The machine learning processed data is presented as cluster curves. In embodiments, the machine learning process comprising detecting the presences or absences of molecules of interest. In further embodiments, the machine learning processes the data as an endpoint detection or a function of time detection. In specific embodiment, the machine learning detection of molecules of interest is a function of time detection. This machine learning process allows for differentiation of the samples into specific category or sub-category.

### EXAMPLES

### Example 1

Data sets have been obtained using the IRIS system applied to the detection Lyme antibodies in human serum. To evaluate human serum binding to Lyme antigen components, a series of Lyme target antigens were spotted on the chip: VoVo, VoBop and Vo4. The in-house Lyme antigen candidate has been the VoVo construct. Additional controls included targets for Babesia (B. microti), anti-human IgM and anti-human IgG spots.

Samples are diluted 1:100 into buffer and first injected into the flow cell for 10 minutes. Gold conjugate targeting human IgM is then injected into the chamber for 10 minutes. Finally, gold conjugate targeting human IgG is injected to confirm human IgG analyte.

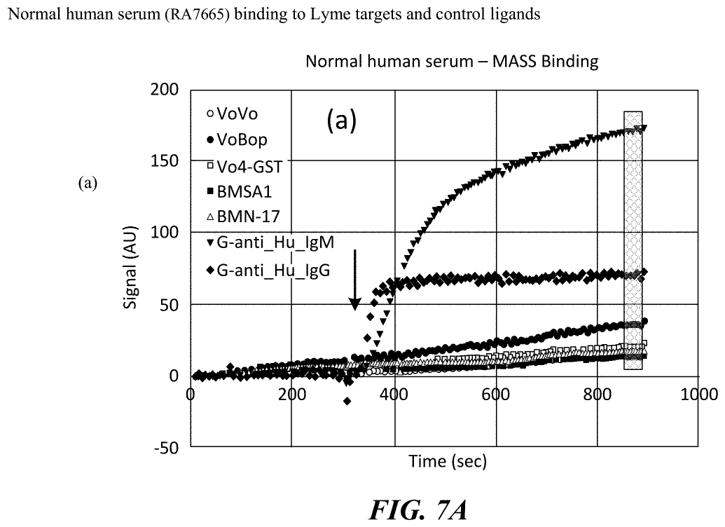

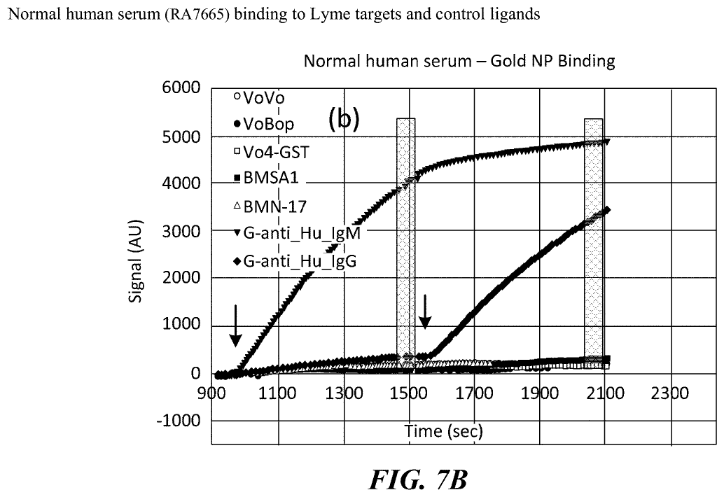

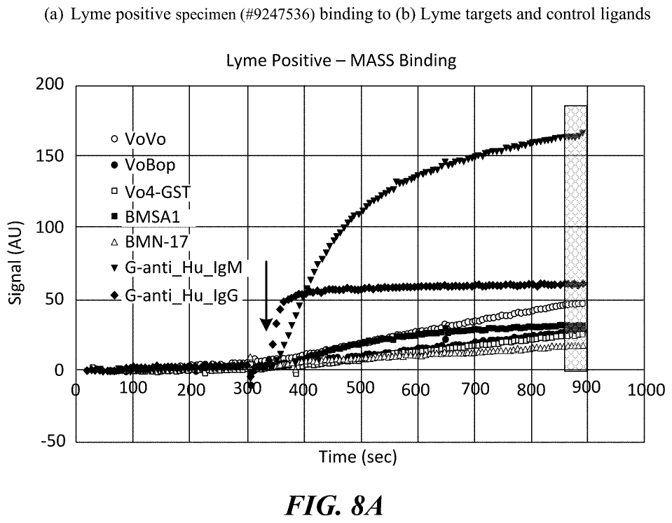

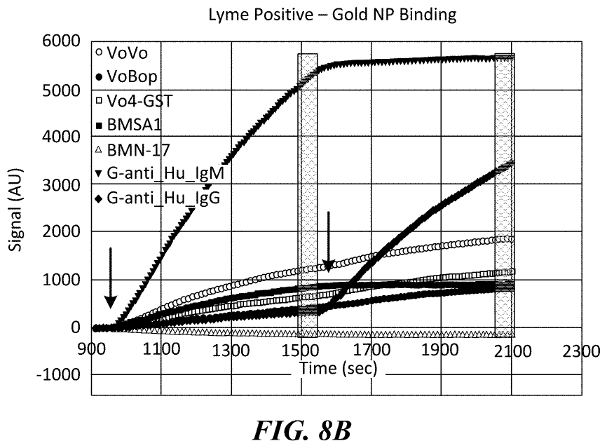

An example normal human serum (NHS) specimen (RA7665) binding to Lyme targets is shown in FIG. 7A and FIG. 7B and Lyme positive specimen (#9247536) binding to Lyme targets is shown in FIG. 8A and FIG. 8B. The highest climbing and second highest climbing lines on the charts show in each of FIG. 7A, FIG. 7B, FIG. 8A, and FIG. 8B represent the binding of the human IgM and IgG, respectively, to the appropriate capture antibodies on the test chip (positive controls).

For comparison, the “mass binding” end-points for the normal human serum and Lyme positive specimen was evaluated, results indicate significant (above background) binding, Table 1.

TABLE 1
End-point values (AU) indicative of mass binding events

| Time(sec) | VoVo | VoBop | Vo4 | BMSA1 | BMN-17 | G-anti_Hu_IgM | G-anti_Hu_IgG |
|---|---|---|---|---|---|---|---|
| NHS | 15.42 | 35.80 | 20.45 | 14.09 | 17.13 | 170.32 | 70.79 |
| Lyme Pos | 45.24 | 26.15 | 23.57 | 30.84 | 16.36 | 162.04 | 59.72 |

Table 1 shows positive control signals for the NHS and Lyme positive are indicated with the anti-human IgG and anti-human IgM capture values. Some specific mass accumulation is observed (above background/control values) with the Lyme positive specimen with the VoVo and BMSA1 (Babesia) constructs. However, some unexpected mass binding is also observed with the VoBop construct and the NHS specimen (this is ameliorated with the addition of the gold nanoparticles in the subsequent steps).

The comparative end-point values after the addition of the gold nanoparticles targeting human IgM was observed, the results are illustrated in Table 2.

TABLE 2
End-point values (AU) for GOLD nanoparticles targeting IgM

| Time(sec) | VoVo | VoBop | Vo4-GST | BMSA1 | BMN-17 | G-anti_Hu_gM | G-anti_Hu_IgG |
|---|---|---|---|---|---|---|---|
| NHS | 194.07 | 51.07 | 142.29 | 79.67 | −9.05 | 3817.19 | 349.93 |
| Lyme | 1173.79 | 386.28 | 622.72 | 811.41 | −110.67 | 4915.27 | 324.44 |

Table 2 illustrates after the addition of gold nanoparticles targeting human IgM, the specific binding to human IgM may be observed and amplified. Here, the end point values for VoVo, Vo4, and BMSA1 antigens are considerably stronger than the example NHS specimen.

Lastly, the comparative results upon the addition of the gold nanoparticles that target human IgG were considered. These results are shown in Table 3.

TABLE 3
End-point values (AU) for GOLD nanoparticles targeting IgG

| Time(sec) | VoVo | VoBop | Vo4-GST | BMSA1 | BMN-17 | G-anti_Hu _IgM | G-anti_Hu_IgG |
|---|---|---|---|---|---|---|---|
| NHS | 231.45 | 198.48 | 177.59 | 326.04 | 66.83 | 4858.82 | 3313.15 |
| Lyme | 1850.65 | 842.38 | 1156.06 | 909.16 | −129.42 | 5640.17 | 3344.95 |

Table 3. After the addition of gold nanoparticles targeting human IgG, the specific binding to human IgG may be observed and amplified. Here, the end point values for VoVo, VoBop, Vo4 and BMSA1 antigens are considerably stronger than the example NHS specimen. It is noted that there is about a 10-fold rise in signal at the anti-human IgG test spot.

For reference purposes, an indirect IgM ELISA which incorporates these two specimens is also shown in Table 4. An approximate ELISA signal with the VoVo antigen for Lyme positive specimen #9247536 was about an 8-fold over the normal human serum sample RA7665 (hour long incubation steps). A more modest reactivity of this specimen to the Babesia antigen, BMSA1, was also observed in an IgM ELISA (data not shown). A similar 6-fold increase in signal is likewise observed at the end-point IgM values with the IRIS system as well as an 8-fold increase in signal at the end-point IgG values (10 minute incubation steps).

We also note that while the ELISA tested for IgM reactivity to the VoVo antigen only, the IRIS system tested for reactivity to VoVo, VoBop, Vo4, BMSA1, BMN17 and included positive controls for human IgG and human IgM. Mass binding and specific human IgM and IgG signals were also recorded. Additional target antigens may also be added in the future. The assay time for the IgM ELISA was approximately 2.25 hours while the assay time for the IRIS was about 35 minutes.

TABLE 4
The IgM ELISA values for the positive and NHS specimen that
were tested with the IRIS system are shown here (highlighted).
IgM ELISA values targeting VoVo: Lyme positive and NHS

| Sample Description | Sample ID | Raw OD₄₅₀ |
|---|---|---|
| SeraCare IgM Positive | 9235031 | 0.949 |
| | 9235555 | 0.527 |
| | 9238472 | 1.708 |
| | 9247534 | 1.625 |
| | 9247536 | 1.958 |
| St. Luke's Lyme | Sample #2 | 2.871 |
| Positive Sera | (⅕ dilution) | |
| InBios NHS | RA7659 | 0.222 |
| | RA7660 | 0.162 |
| | RA7661 | 0.134 |
| | RA7662 | 0.196 |
| | RA7664 | 0.119 |
| | RA7665 | 0.246 |
| | RA7666 | 0.247 |
| | RA7667 | 0.220 |

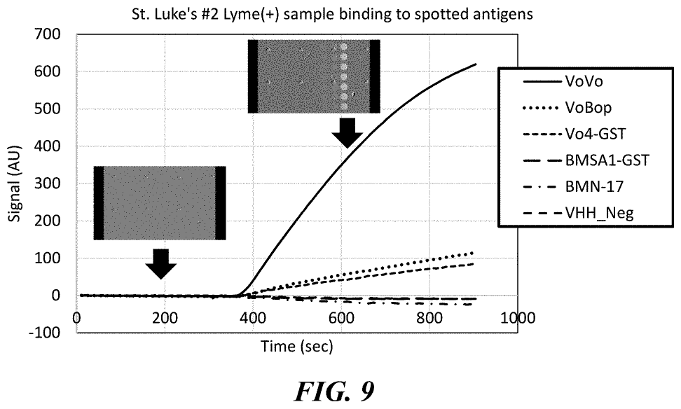

We also point out that stronger specimens in ELISA should also be expected to provide stronger signals in the IRIS system. For instance, the St. Luke's sample #2 was tested at a 1:100 dilution to monitor for reactivity to VoVo in the IRIS system and the results for the mass binding are shown in FIG. 9. As depicted in FIG. 9, the mass binding of the St. Luke's #2 specimen to the VoVo antigen is markedly stronger—with signals exceeding about 600 AU in less than 10 minutes.

### Conclusions from Example 1

The use of the IRIS platform provides the unique ability to screen single specimens against a multiplicity of targets and separately detect (1) mass binding and (2) amplified specific signal with gold nanoparticle conjugates. The test chip and reagents may be provided as room temperature stable components and do not suffer from quenching or photobleaching (as is evident with fluorescent labels). Assay time is minimal (about 30 minutes) to test for mass binding, specific IgM and specific IgG signals. Preliminary example data sets for normal human serum and Lyme positive serum specimens have been provided and appear to correlate to expected values obtained via ELISA. Additional screening has been performed with other infectious disease types (e.g., Zika) with similar correlative results as obtained with ELISA testing.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### REFERENCES

1. Avci, O., Ünlü, N. L., Özkumur, A. Y., & Ünlü, M. S. (2015). Interferometric Reflectance Imaging Sensor (IRIS)—A Platform Technology for Multiplexed Diagnostics and Digital Detection. Sensors, 15, 17649-17665.
2. Needham, J. (2019). Multiplexed Antibody Kinetics using the Interferometric Reflectance Imaging Sensor. Dissertation (Boston University).
3. Needham, J. W., Lortlar Ünlü, N., Yurdakul, C., & Ünlü, M. S. (2019). Interferometric Reflectance Imaging Sensor (IRIS) for Molecular Kinetics with a Low-Cost, Disposable Fluidic Cartridge. Springer Chapter—pending, TBD.
4. Özkumur, E., Needham, J. W., Bergstein, D. A., Gonzalez, R., Cabodi, M., Gershoni, J. M., . . . Ünlü, M. S. (2008). Label-free and dynamic detection of biomolecular interactions for high-throughput microarray applications. Proc Natl Acad Sci USA, 105(23), 7988-7992.
5. Trueb, J. (2018). Enabling and understanding nanoparticle surface binding assays with interferometric imaging. Dissertation. Boston, Mass.: Boston University.

1. An apparatus comprising:
a. an interferometric based imaging sensor (IRIS);
b. multiple distinct target compatible materials bound to a functionalized chip surface;
c. a stabilized chip surface in contact with a fluidic cartridge, wherein said cartridge is stable at room temperature for an extended period of time;
d. the fluidic cartridge, optionally, in contact with a fluidics system;
e. a cassette housing, wherein said housing provides for at least one of manipulation, protection, and labelling of the fluidic cartridge; and
f. one or more stabilized and/or lyophilized conjugate nanoparticles, wherein said nanoparticles provide for subsequent signal detection.

2. A method of using the apparatus of claim 1, comprising:
a. preparing a heterogeneous biological sample by diluting the biological sample in an appropriate buffer;
b. contacting the sample with the chip; and
c. imaging material mass accumulation resulting from binding between at least one of the multiple distinct target compatible materials and at least one component of the sample.

3. The method of using the apparatus of claim 2, comprising:
a. injecting labeled nanoparticles after the imaging material mass accumulation in a wide field-of-view to observe at least one of reflection and scattering of light from the accumulation of nanoparticles at localized spots on the chip surface;
b. sequentially injecting uniquely labeled nanoparticles to provide separate, step-wise information about the specimen status; and, optionally,
c. simultaneously injecting diluted biological samples pre-mixed with labeled nanoparticles.

4. The apparatus of claim 1, further comprising a room temperature stabilized fluidic cartridge system.

5. An appropriate buffer for diluting and running a heterogeneous biological sample for use with the apparatus of claim 1.

6. A method of using the apparatus of claim 3, comprising step-wise injections of a heterogeneous biological sample and labeled nanoparticles in order to sequentially detect useful information about the specimen (e.g., IgM antibody content followed by IgG antibody content).

7. A method of using the apparatus of claim 3, comprising the use of disease specific analytes to detect direct mass accumulation followed by more specific step-wise conjugate nanoparticles to indicate disease status for real-time and endpoint binding measurements.

8. A method of using the apparatus of claim 3, comprising the step-wise detection of the specimen binding events, wherein mass binding is followed by conjugated nanoparticle binding.

9. A method of using the apparatus of claim 3, comprising detecting of an antibody isotype.

10. The apparatus of claim 1, further comprising a room temperature stabilized and/or lyophilized conjugate nanoparticle system to be incorporated with step-wise detection of the specimen binding events for the detection of a specific antigen or nucleic acid present in the specimen.

11. The apparatus of claim 1, further comprising a series of positive and negative control analytes to indicate the presence of the diluted specimen and to monitor for non-specific binding.

12. A method for imaging material mass accumulation followed by more specific gold nanoparticle accumulation observed in the wide-field.

13. An apparatus comprising one or more of:
a. multiple distinct target compatible materials bound to a chip, wherein said materials target tick borne infections;
b. multiple distinct target compatible materials bound to a chip, wherein said materials target flavivirus and alphavirus infections;
c. multiple distinct target compatible materials bound to a chip, wherein said materials target flu, HIV-1, HIV-2, HBV, and HCV;
d. multiple distinct target compatible materials bound to a chip, wherein said materials target known allergens;
e. a series of target analytes specific for Lyme infection including VoVo, VoBop, Vo4, OspC variants, DbpA, DbpB, VIsE, FlaB and C6 bound to the chip surface;
f. a series of target analytes specific for Babesia infection including BMSA1 and BMN1-17 bound to the chip surface;
g. a series of target analytes specific for dengue and zika infection including dengue NS1 and envelope proteins
h. a series of antibodies targeting dengue NS1 for the detection of circulating dengue NS1 in a patient specimen including 528.1133, 528.292, 528.1299, 1010.511 and 1010.522; and
i. a series of target analytes specific for chikungunya infection, including chikungunya envelope proteins

14. A method comprising one or more of:
a. analysis of binding events that incorporate at least of one of dynamic and endpoint measurements;
b. cut-off thresholds to determine positivity based on one or multiple target binding events to a target;
c. cut-off thresholds that incorporate one or multiple targets;
d. cut-off thresholds that incorporate measurements from mass accumulation and step-wise addition of conjugated nanoparticles, wherein said measurements are incorporated either individually or jointly; and
e. machine learning applied to categorizing specimens (e.g., positive or negative, IgM positive, antigen positive, etc.) based upon at least one of real-time or endpoint measurements with at least one of mass and step-wise addition of conjugated nanoparticles, and wherein a training set is used to train and properly classify specimens according to the desired status.
